(19) United States
(12) Patent Application Publication
(10) Pub. No.: US 2026/0240557 A1
(43) Pub. Date: Aug. 20, 2026

(54) RECAPTURABLE FUNNEL CATHETERS, AND ASSOCIATED SYSTEMS AND METHODS

(71) Applicant: Stryker Corporation, Portage, MI (US)

(72) Inventors: John Coleman Thress, Capistrano Beach, CA (US); Benjamin Edward Merritt, San Clemente, CA (US); Jacob F. Louw, Carlsbad, CA (US); Jacqueline Macias, Fullerton, CA (US); Brian Michael Strauss, San Clemente, CA (US); Marcus Ian Tambongco Zozulenko, Arleta, CA (US); Christopher Andrew Zikry, Northridge, CA (US); Cheng Lance Ian, Lake Forest, CA (US); Juan Valencia, Fullerton, CA (US)

(21) Appl. No.: 19/462,487

(22) Filed: Jan. 28, 2026

Related U.S. Application Data
(63) Continuation of application No. 17/339,663, filed on Jun. 4, 2021, now Pat. No. 12,558,112.
(60) Provisional application No. 63/035,605, filed on Jun. 5, 2020.

(51) Int. Cl. A61B 17/221; A61B 17/00

(52) U.S. Cl. CPC A61B 17/221; A61B 2017/00292; A61B 2017/2215

(57) ABSTRACT

Systems and methods for the intravascular treatment of clot material within a blood vessel of a human patient are disclosed herein. In one embodiment, a funnel catheter assembly includes an outer shaft and an inner shaft extending through and coaxial with the outer shaft. An expandable funnel can be coupled to a distal portion of the inner shaft. The funnel catheter assembly further includes a control assembly operably coupled to the proximal portion of the outer shaft and configured to move the outer shaft between a first position and a second position. In the first position, the outer shaft is positioned at least partially over the funnel to constrain the funnel in a compressed state. In the second position, the outer shaft is retracted proximally relative to the funnel such that the funnel can expand to an expanded state.

### CROSS-REFERENCE TO RELATED APPLICATION

This is a continuation of U.S. Patent Application No. 17/339,663, filed June 4, 2021, which claims the benefit of U.S. Provisional Patent Application No. 63/035,605, filed June 5, 2020, each of which is herein incorporated by reference in its entirety.

### TECHNICAL FIELD

The present technology generally relates to systems, methods, and devices for embolic protection during procedures for extracting thrombi from blood vessels of human patients.

### BACKGROUND

Thrombosis is the local coagulation or clotting of the blood in a part of the circulatory system, and a thrombus is a blood clot formed in situ within the vascular system. A venous thrombus is a blood clot that forms within a vein. A common type of venous thrombosis is a deep vein thrombosis (DVT), which is the formation of a blood clot within a deep vein (e.g., predominantly in the legs). Nonspecific signs of a thrombosis may include pain, swelling, redness, warmness, and engorged superficial veins.

If the thrombus breaks off (embolizes) and flows towards the lungs, it can become a life-threatening pulmonary embolism (PE) (e.g., a blood clot in the lungs). In addition to the loss of life that can arise from PE, DVT can cause significant health issues such as post thrombotic syndrome, which can cause chronic swelling, pressure, pain, and ulcers due to valve and vessel damage. Further, DVT can result in significant health-care costs either directly or indirectly through the treatment of related complications and inability of patients to work.

Three processes are believed to result in venous thrombosis. First is a decreased blood flow rate (venous stasis), second is an increased tendency to clot (hypercoagulability), and the third is changes to the blood vessel wall. DVT formation typically begins inside the valves of the calf veins where the blood is relatively oxygen deprived, which activates certain biochemical pathways. Several medical conditions increase the risk for DVT, including diabetes, cancer, trauma, and antiphospholipid syndrome. Other risk factors include older age, surgery, immobilization (as with bed rest, orthopedic casts, and sitting on long flights), combined oral contraceptives, pregnancy, the postnatal period, and genetic factors. The rate of DVT increases dramatically from childhood to old age and, in adulthood, about 1 in 1,000 adults develop DVT annually.

Although current devices and methods of prevention and/or treatment of DVT exist, there are a number of shortcomings that have yet to be resolved, such as high incidence of DVT re-occurrence, use of devices not designed to remove large clot volumes, and/or complicated treatments involving multiple treatment devices and/or pharmaceuticals. Accordingly, new devices, systems, and methods of treating thrombus, and particularly DVT are desired.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially transparent and partially cross-sectional side view of a funnel catheter assembly in accordance with embodiments of the present technology.

FIGS. 2A and 2B are side views of the funnel catheter assembly in a first position and a second position, respectively, in accordance with embodiments of the present technology.

FIG. 3 is a side view of the funnel catheter assembly in the first position with a dilator inserted therethrough in accordance with embodiments of the present technology.

FIG. 4 is a side view of the funnel catheter assembly including a non-self-expanding funnel in accordance with embodiments of the present technology.

FIGS. 5–8 are schematic views illustrating various thrombectomy techniques for removing a thrombus from a blood vessel of a human patient utilizing the funnel catheter assembly in accordance with embodiments of the present technology.

FIGS. 9A-9C are enlarged, partially transparent side and side cross-sectional views, respectively, of a portion of a funnel catheter assembly in accordance with additional embodiments of the present technology.

FIGS. 10A–10C are side views of the funnel catheter assembly of FIGS. 9A and 9B in a sheathed position, a partially-unsheathed or intermediate position, and an unsheathed position, respectively, in accordance with embodiments of the present technology.

FIG. 11 is a flow diagram of a process or method for operating the funnel catheter assembly during an intravascular procedure in accordance with embodiments of the present technology.

### DETAILED DESCRIPTION

The present technology is generally directed to methods and systems for removing clot material (e.g., a thrombus) from a blood vessel of a human patient. More particularly, the present technology is directed to funnel catheter assemblies configured to provide embolic protection during a clot removal or other intravascular procedure. In some embodiments, a funnel catheter assembly includes an outer shaft and an inner shaft extending through and coaxial with the outer shaft. An expandable funnel, such as a self-expanding funnel, can be coupled to a distal portion of the inner shaft. The funnel catheter assembly further includes a control assembly operably coupled to the proximal portion of the outer shaft. The funnel catheter assembly can be actuated by an operator (e.g., a physician) to move the outer shaft between a first position and a second position. In the first position, the outer shaft is positioned at least partially over the funnel to constrain the funnel in a compressed state. In the second position, the outer shaft is retracted proximally relative to the funnel such that the funnel can expand to an expanded state. Accordingly, the funnel catheter assembly enables the funnel to be unsheathed and sheathed during an intravascular procedure via the movement of the outer shaft between the first and second positions.

In one aspect of the present technology, the control assembly is operable to compress the funnel after it has been expanded within a blood vessel of a patient. This can permit the funnel catheter assembly to be repositioned within the blood vessel without fully withdrawing the funnel catheter assembly from the patient. Similarly, the funnel catheter assembly can be fully withdrawn from the patient (e.g., at the conclusion of a thrombectomy procedure) in the first position with the funnel compressed inside the outer shaft. Thus, the funnel catheter assembly is configured to inhibit or even prevent the funnel from contacting the wall of the blood vessel during movement of the funnel catheter assembly within the blood vessel. This can help inhibit injury/damage to the patient that could otherwise be caused by the moving the funnel through a blood vessel or an associated organ in the expanded state.

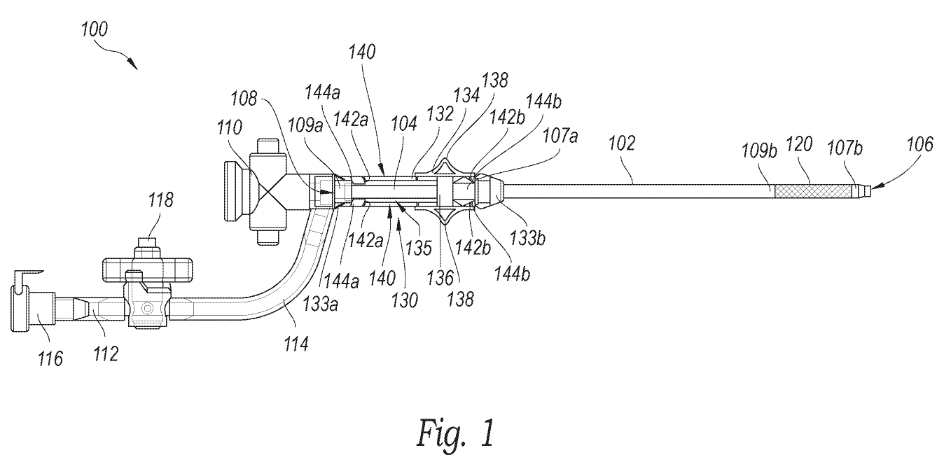

Although many of the embodiments are described below with respect to devices, systems, and methods for treating vascular thrombi (e.g., deep vein thrombosis (DVT)), other applications and other embodiments in addition to those described herein are within the scope of the technology (e.g., intravascular procedures other than the treatment of emboli, intravascular procedures for treating cerebral embolism, intravascular procedures for treating pulmonary embolism, etc.). In general, for example, the devices, systems, and methods of the present technology can be used to extract any formation of material in a vessel (e.g., a venous or arterial vessel), such as cancerous growths, vegetation, etc. Additionally, several other embodiments of the technology can have different configurations, states, components, or procedures than those described herein. Moreover, it will be appreciated that specific elements, substructures, advantages, uses, and/or other features of the embodiments described with reference to FIGS. 1–11 can be suitably interchanged, substituted or otherwise configured with one another in accordance with additional embodiments of the present technology. Furthermore, suitable elements of the embodiments described with reference to FIGS. 1–11 can be used as standalone and/or self-contained devices. A person of ordinary skill in the art, therefore, will accordingly understand that the technology can have other embodiments with additional elements, or the technology can have other embodiments without several of the features shown and described below with reference to FIGS. 1–11.

With regard to the terms "distal" and "proximal" within this description, unless otherwise specified, the terms can reference a relative position of the portions of a catheter subsystem with reference to an operator and/or a location in the vasculature. Also, as used herein, the designations "rearward," "forward," "upward," "downward," etc. are not meant to limit the referenced component to use in a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### Selected Embodiments of Funnel Catheter Assemblies

FIG. 1 is a partially transparent and partially cross-sectional side view of a funnel catheter assembly 100 in accordance with embodiments of the present technology. In the illustrated embodiment, the funnel catheter assembly 100 includes an elongate outer shaft 102 and an elongate inner shaft 104 coaxial with the outer shaft 102. The outer and inner shafts 102, 104 ("shafts 102, 104") can also be referred to as sheaths, catheters, hollow members, and so on. The outer shaft 102 defines a lumen 106 and includes a proximal portion 107a and a distal portion 107b. The inner shaft 104 extends through the lumen 106 of the outer shaft 102 and similarly defines a lumen 108 and includes a proximal portion 109a and a distal portion 109b. The proximal portions 107a and 109a can each terminate at a proximal end/terminus, and the distal portions 107b and 109b can each terminate at a distal end/terminus. The shafts 102, 104 can be elastic and/or flexible and can have any suitable length and diameter.

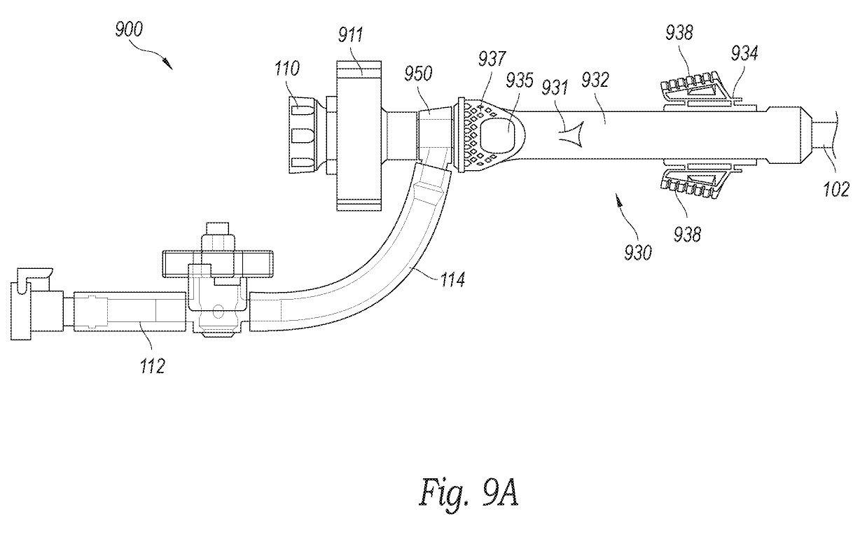

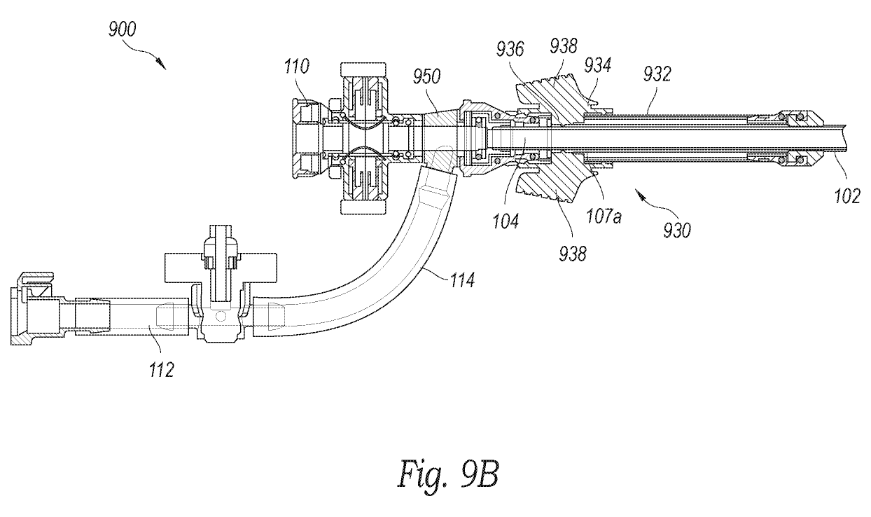

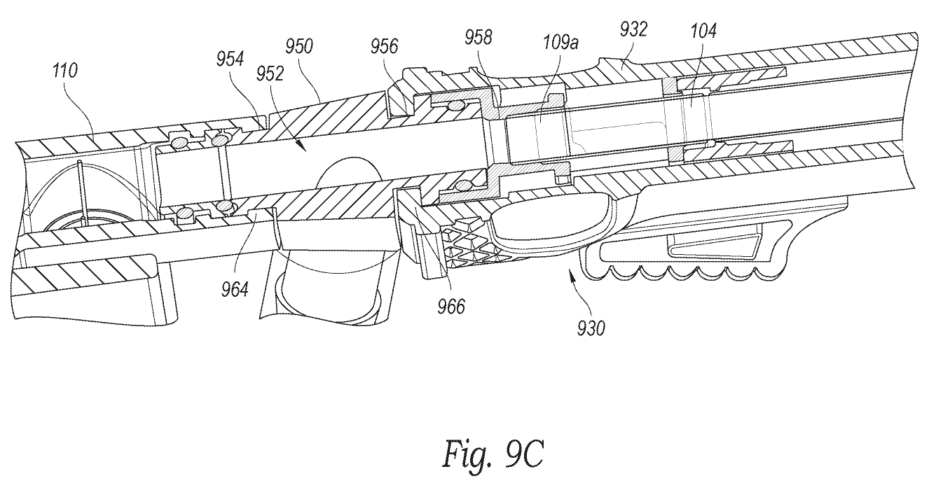

In the illustrated embodiment, the funnel catheter assembly 100 further includes a sealable hub 110 coupled to the proximal portion 109a of the inner shaft 104. In some embodiments, as described in greater detail below with reference to FIGS. 9A–9C, the sealable hub 110 can be rotatable coupled to the inner shaft 104 such that the sealable hub 110 can rotate independently of the inner shaft 104. The sealable hub 110 is configured to allow access to the lumen 108 of the inner shaft 104 and can be self-sealing and/or can comprise a self-sealing seal. For example, in the illustrated embodiment the sealable hub 110 is a hemostasis valve that is configured to maintain hemostasis (e.g., during a thrombus extraction procedure) by inhibiting or even preventing fluid flow in the proximal direction through the sealable hub 110 as, for example, various components (e.g., dilator assemblies, thrombus extraction devices, etc.) are inserted through the sealable hub 110 to be delivered through the inner shaft 104 to a treatment site in a blood vessel. More specifically, the sealable hub 110 can be a valve of the type disclosed in U.S. Patent Application No. 16/117,519, filed August 30, 2018, and titled "HEMOSTASIS VALVES AND METHODS OF USE," which is incorporated herein by reference in its entirety. The sealable hub 110 can include one or more buttons or actuators that enable an operator to selectively seal/unseal the sealable hub 110.

The funnel catheter assembly 100 can further include an aspiration port 112 connected to the sealable hub 110 (e.g., to a side port of the sealable hub 110) and/or the inner shaft 104 (e.g., to the proximal portion 109a of the inner shaft 104) via, for example, a connecting tube 114. The aspiration port 112 can be connected to a syringe connector 116 that can be selectively coupled to a syringe or other aspiration device, or the aspiration port 112 can be connected to other suitable elements. In some embodiments, the funnel catheter assembly 100 includes a fluid control device 118 configured to selectively fluidly connect the aspiration port 112 to the lumen 108 of the inner shaft 104. In the illustrated embodiment, the fluid control device 118 is a stopcock operably coupled to the connecting tube 114 between the lumen 108 of the inner shaft 104 and the aspiration port 112. In other embodiments, the fluid control device 118 can be a clamp or another suitable valve. In some embodiments, a vacuum source (not shown; e.g., a syringe) can be coupled to the syringe connector 116 and used to aspirate the lumen 108 of the inner shaft 104. In some embodiments, as described in greater detail below with reference to FIGS. 9A–9C, the aspiration port 112 and the connecting tube 114 can be rotatable relative to the sealable hub 110 and/or the inner shaft 104.

In the illustrated embodiment, a funnel 120 is coupled to the distal portion 109b of the inner shaft 104. As described in greater detail below, in operation of the funnel catheter assembly 100 during an intravascular procedure, the funnel 120 is configured to expand (e.g., radially expand) into apposition with a blood vessel and/or other bodily lumen (e.g., of an organ) and act as a proximal or distal thrombus/embolic protection device that inhibits any thrombus from moving past the funnel 120 and embolizing in an unwanted location (e.g., the right heart, the pulmonary arteries, another arterial space, etc.). The funnel 120 can be fused to the distal portion 109b of the inner shaft 104, and/or attached to the inner shaft 104 via welding, adhesives, fasteners, etc. In FIG. 1, the funnel catheter assembly 100 is in a first position/state/configuration in which the distal portion 107b of the outer shaft 102 extends beyond the distal portion 109b of the inner shaft 104 such that the funnel 120 is at least partially (e.g., entirely) positioned within the lumen 106 of the outer shaft 102. The funnel 120 can be configured to expand (e.g., self-expand) and, accordingly, the funnel 120 can be in a first, constrained position/state/configuration when the funnel catheter assembly 100 is in the first position. In some embodiments, the funnel 120 can be formed from at least one of a castellated nitinol braid, a nitinol braided stent, a laser cut nitinol, a laser cut polymer tube, an injection molded polymeric structure, or an inflatable balloon. In some embodiments, the funnel 120 can comprise a mesh having a pore size sufficiently small to prevent the passage of thrombus through the pores of the mesh. In some embodiments, the funnel 120 can be permeable to blood. In some embodiments, the funnel 120 can include a covering over at least a portion thereof that is permeable or non-permeable to blood.

In the illustrated embodiment, the proximal portion 107a of the outer shaft 102 is operably coupled to a control assembly 130. The control assembly 130 is operable to move the outer shaft 102 distally and proximally relative to the inner shaft 104 to constrain and release the funnel 120 from within the lumen 106 of the outer shaft 102. More specifically, in the illustrated embodiment the control assembly 130 includes (i) a housing 132 having a proximal portion 133a and a distal portion 133b, and (ii) an actuation member 134 operably/movably coupled to the housing 132. The housing 132 defines a lumen 135 extending between the proximal and distal portions 133a, b thereof. The proximal portion 133a of the housing 132 can be coupled to the sealable hub 110. In some embodiments, the proximal portion 133a of the housing 132 is integrally formed with the sealable hub 110.

In the illustrated embodiment, the actuation member 134 includes a body portion 136 positioned within the lumen 135 of the housing 132 and coupled to the proximal portion 107a of the outer shaft 102 via, for example, adhesive, fasteners, welding, etc. The actuation member 134 further includes one or more grip members 138 extending from the body portion 136 to outside of the lumen 135. More specifically, the grip members 138 can extend through corresponding slots 140 formed in/along the housing 132. The housing 132 can define a proximal terminus 142a and a distal terminus 142b for each of the slots 140. In some embodiments, the control assembly 130 can include one, or more than the illustrated two of the grip members 138 and corresponding slots 140.

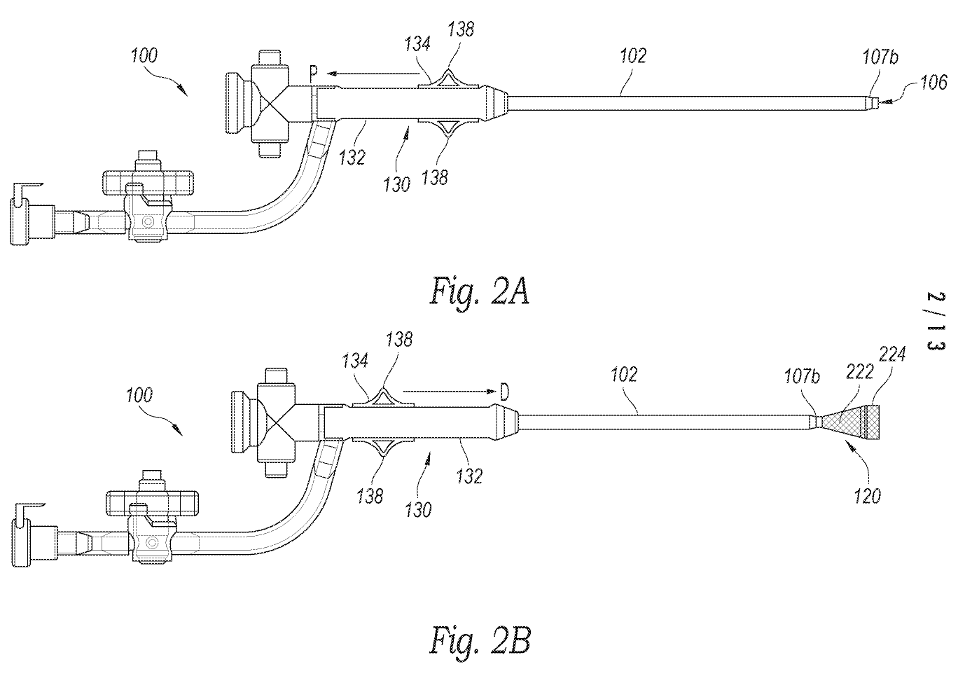

In operation, an operator (e.g., a physician) can slide the actuation member 134 along the housing 132 to distally advance and proximally retract the outer shaft 102 relative to the inner shaft 104 to constrain and release the funnel 120, respectively. More specifically, FIGS. 2A and 2B are side views of the funnel catheter assembly 100 in the first position and in a second position, respectively, in accordance with embodiments of the present technology. Referring to FIGS. 1–2B together, to move the funnel catheter assembly 100 from the first position (FIGS. 1 and 2A) to the second position (FIG. 2B), the operator can grip the grip members 138 of the actuation member 134 and slide the grip members 138 proximally (e.g., in the direction of arrow P in FIG. 2A) relative to the housing 132 to drive the body portion 136 through the lumen 135 to thereby drive the outer shaft 102 proximally relative to the inner shaft 104. The funnel 120 is released/unsheathed from within the lumen 106 of the outer shaft 102 as the distal portion 107b of the outer shaft 102 moves proximally past the funnel 120, thereby allowing the funnel 120 to expand. In some embodiments, the actuation member 134 can abut the proximal termini 142a of the slots 140 in the second position such that the housing 132 inhibits further proximal movement of the outer shaft 102.

In some embodiments, the distal terminus of the outer shaft 102 is positioned at or proximal of the distal terminus of the inner shaft 104 in the second position such that the funnel 120 is fully released from (e.g., positioned fully outside of) the lumen 106 of the outer shaft 102. In other embodiments, the distal terminus of the outer shaft 102 can be positioned distal of the distal terminus of the inner shaft 104 in the second position such that the funnel 120 is only partially released from the lumen 106 of the outer shaft 102. Moreover, as shown in FIG. 2B, the funnel 120 can have a conically shaped portion 222 (e.g., a truncated-conically shaped portion) and a cylindrical portion 224 once expanded. In other embodiments, the funnel 120 can have other suitable shapes. For example, in some embodiments the funnel 120 can be inverted relative to the embodiment shown in FIG. 2B. That is, the cylindrical portion 224 of the funnel 120 can be coupled to the distal portion 109b of the inner shaft 104 while the conically shaped portion 222 extends distally from the cylindrical portion 224.

To move the funnel catheter assembly 100 from the second position (FIG. 2B) to the first position (FIGS. 1 and 2A), the operator can grip the grip members 138 of the actuation member 134 and slide the grip members 138 distally (e.g., in the direction of arrow D in FIG. 2B) relative to the housing 132 to drive the body portion 136 through the lumen 135 to thereby drive the outer shaft 102 distally relative to the inner shaft 104. The funnel 120 is captured/sheathed in the lumen 106 of the outer shaft 102 as the distal portion 107b of the outer shaft 102 moves distally over the funnel 120, thereby collapsing/compressing the funnel 120 within the lumen 106. In some embodiments, the actuation member 134 can abut the distal termini 142b of the slots 140 in the first position such that the housing 132 inhibits further distal movement of the outer shaft 102.

In some embodiments the actuation member 134 is configured to be releasably secured/locked to the housing 132 in the first and second positions to inhibit or even prevent unintended movement of the actuation member 134. For example, referring again to FIG. 1, the housing 132 of the control assembly 130 can include (i) proximal engagement members 144a (e.g., tabs, protrusions, etc.) configured to engage the actuation member 134 in the first position and (ii) distal engagement members 144b configured to engage the actuation member 134 in the second position. More specifically, in some embodiments the proximal and distal engagement members 144a, b can mate with corresponding slots/grooves in the actuation member 134 (e.g., formed in/on the grip members 138) to releasably secure the actuation member 134 in the first or second positions via a snap fit arrangement.

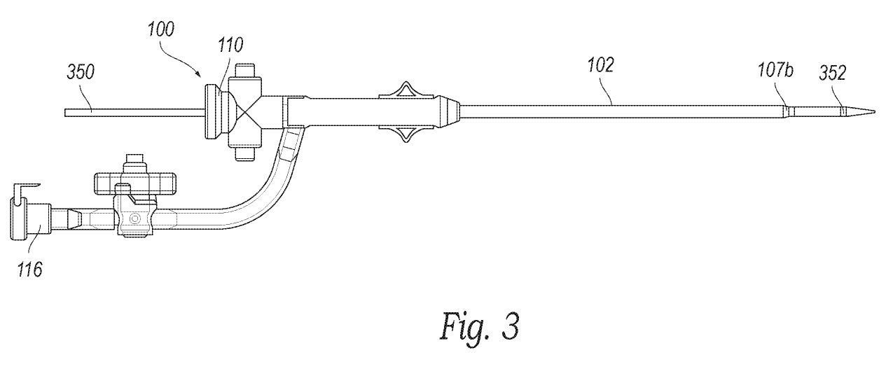

In some embodiments, the inner shaft 104 is sized to slidably receive one or more medical instruments inserted through the sealable hub 110 during an intravascular procedure using the funnel catheter assembly 100, such as a thrombectomy procedure. FIG. 3, for example, is a side view of the funnel catheter assembly 100 in the first position with a dilator 350 inserted through the sealable hub 110 and the lumen 108 of the inner shaft 104 (FIG. 1) in accordance with embodiments of the present technology. The dilator 350 can extend entirely through the inner shaft 104 and past the distal terminus of the distal portion 107b of the outer shaft 102 such that a distal tip 352 (e.g., an atraumatic tip) is positioned beyond the distal terminus of the outer shaft 102.

In some embodiments, the dilator 350 and the funnel catheter assembly 100 together define an introducer assembly that can be inserted into a patient (e.g., a human patient) and subsequently used to introduce intravascular medical devices into the patient. For example, the dilator 350 and the funnel catheter assembly 100 can be inserted into and advanced together through a blood vessel of the patient to a target location in the blood vessel. The dilator 350 can then be retracted proximally through the funnel catheter assembly 100, and the funnel catheter assembly 100 can be moved to the second position to expand the funnel 120 at the target location.

Referring to FIGS. 1–3 together, in one aspect of the present technology the inner shaft 104 is the "working" shaft of the funnel catheter assembly 100 that can be aspirated and/or receive various medical components (e.g., the dilator 350). In contrast, the outer shaft 102 is used to compress/expand the funnel 120. Accordingly, the shafts 102, 104 can be sized to maximize the size of the inner shaft 104 to permit larger-sized components to be inserted therethrough, or to provide greater aspiration force/power. For example, the outer shaft 102 can have an inner diameter that is only slightly greater than an outer diameter of the inner shaft 104. In some embodiments, the inner shaft 104 can have an outer diameter of at least 10 French, at least 12 French, at least 14 French, at least 18 French, at least 20 French, at least 22 French, at least 26 French, greater than 26 French, between 10 French and 26 French, between 14 French and 24 French, between15 French and 21 French, between 16 French and 22 French, and/or any other or intermediate size. In some embodiments, the lumen 108 of the inner shaft 104 can have an internal diameter of at least 2 French, at least 10 French, at least 14 French, at least 18 French, at least 20 French, at least 22 French, between 11 French and 12 French, between 10 French and 22 French, between 14 French and 21 French, between 16French and 20 French, and/or any other or intermediate size.

In another aspect of the present technology, the control assembly 130 is operable, via the movement of the actuation member 134 from the second position to the first position, to compress the funnel 120 after it has been expanded within a blood vessel. This can permit the funnel catheter assembly 100 to be repositioned within the blood vessel without fully withdrawing the funnel catheter assembly 100 from the patient. For example, after (i) introducing the funnel catheter assembly 100 into the blood vessel with the dilator 350 and (ii) removing the dilator 350, the funnel catheter assembly 100 can be repositioned proximally simply by moving the funnel catheter assembly 100 back to the first position to collapse the funnel 120 and then retracting the funnel catheter assembly 100 proximally. To reposition the funnel catheter assembly 100 distally, the dilator 350 can be reinserted and the funnel catheter assembly 100 pushed proximally in the first position with the funnel 120 compressed. Similarly, the funnel catheter assembly 100 can be fully withdrawn from the patient (e.g., at the conclusion of a thrombectomy procedure) in the first position with the funnel 120 compressed in the lumen 106 of outer shaft 102. Thus, the funnel catheter assembly 100 is configured to inhibit or even prevent the funnel 120 from contacting the wall of the blood vessel during advancement/withdrawal. This can help inhibit injury/damage to the patient that could otherwise be caused by the moving the funnel 120 through the blood vessel or an associated organ in the expanded state.

In some embodiments, the funnel catheter assembly 100 and/or methods of operating the funnel catheter assembly 100 can include some features the same as or similar to the thrombectomy systems (e.g., the introducer assemblies) described in detail in (i) U.S. Patent No. 9,700,332, filed September 16, 2016, and titled "INTRAVASCULAR TREATMENT OF VASCULAR OCCLUSION AND ASSOCIATED DEVICES, SYSTEMS, AND METHODS," and/or (ii) U.S. Patent No. 10,098,651, filed April 26, 2017, and titled "DEVICES AND METHODS FOR TREATING VASCULAR OCCLUSION," both of which are incorporated herein by reference in their entirety.

In other embodiments, the control assembly 130 can be configured to drive the distal and proximal movement of the outer shaft 102 in other manners. For example, the actuation member 134 can comprise a rotatable member (e.g., a ring gear, corkscrew, rotatable handle, etc.) coupled to the outer shaft 102. In some embodiments, the rotatable member can be a ratcheting member that is rotatable to a plurality of discreet positions between the first and second positions.

Similarly, in other embodiments the control assembly 130 can be operably coupled to the inner shaft 104 rather than the outer shaft 102. Accordingly, operation of the control assembly 130 can move the inner shaft 104 and the sealable hub 110 relative to the outer shaft 102 to move the funnel catheter assembly 100 between the first and second positions to constrain/compress and release/expand the funnel 120, respectively.

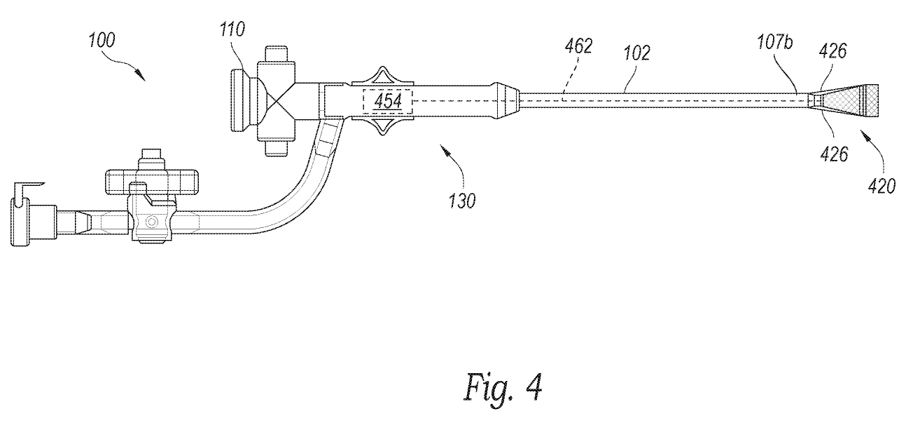

In yet other embodiments, the funnel 120 can be operably coupled to the outer shaft 102 such that movement of the outer shaft 102 between the first and second positions expands the funnel 120. More specifically, for example, FIG. 4 is a side view of the funnel catheter assembly 100 including a non-self-expanding funnel 420 in accordance with embodiments of the present technology. In some embodiments, the funnel 420 can have a similar shape as the funnel 120 described in detail above with reference to FIGS. 1–3, but can be formed of one or more non-self-expanding materials such as metal (e.g., a non-heat-activated metal), plastic, fiber, polymer, etc. In the illustrated embodiment, the funnel 420 is coupled to (i) the distal portion 109b of the inner shaft 104 (FIG. 1) and (ii) the distal portion 107b of the outer shaft 102 via a plurality of flexible tethers 426. The tethers 426 are configured (e.g., shaped, sized, and/or positioned) such that movement of the control assembly 130 from the first position to the second position pulls the tethers 426 to thereby pull and expand the funnel 420 as it is unsheathed from within the outer shaft 102.

In some embodiments, the funnel catheter assembly 100 can include one or more features for actuating/manipulating the funnel 420 (or the funnel 120 described in detail with reference to FIGS. 1–3). For example, the control assembly 130 can include an actuation member 454 (shown schematically) operably coupled to the funnel 420 via one or more control lines 462 (e.g., wires, tethers, rigid members etc.). The actuation member 454 can be a slider, rotatable member, or other component configured to exert a force on the funnel 420 via the control lines 462. In some embodiments, the control lines 462 can be asymmetrically/eccentrically coupled to the funnel 420 such that actuation of the actuation member 454 bends the funnel 420 away from a longitudinal axis of the shafts 102, 104. In one aspect of the present technology, this arrangement can be used to help steer the funnel 420 into a tortuous region of a patient (e.g., a tortuous vessel, the left atrial appendage (LAA), etc.) that may otherwise be difficult to position the funnel 420 in. Additionally or alternatively, the funnel catheter assembly 100 can include one or more components (e.g., pull wires) for steering (e.g., bending, deflecting, etc.) the outer shaft 102 and/or the inner shaft 104 to facilitate positioning of the funnel 420.

### Selected Embodiments of Procedures Utilizing Funnel Catheter Assemblies for Embolic Protection

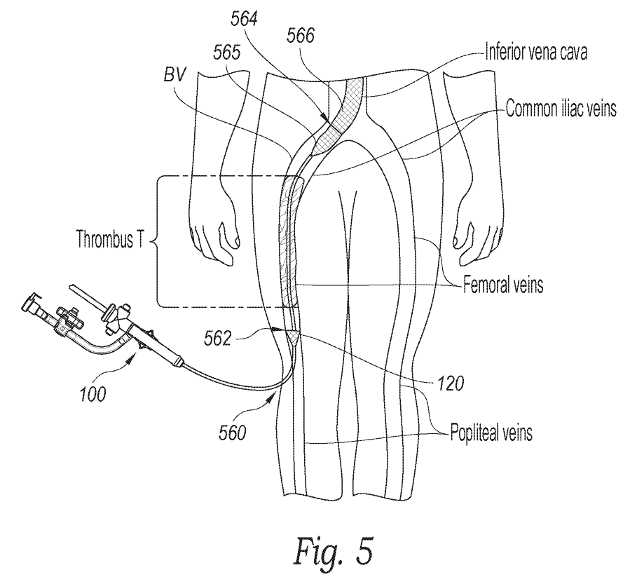

Referring to FIGS. 1–4 together, the funnel catheter assembly 100 can be used in a myriad of procedures to capture thrombi and inhibit the thrombi from embolizing in portions of a patient's vasculature. For example, FIGS. 5–8 are schematic views illustrating various thrombectomy techniques for removing a thrombus T from a blood vessel BV of a human patient utilizing the funnel catheter assembly 100 in accordance with various embodiments of the present technology.

Referring first to FIG. 5, in some embodiments the thrombus T (e.g., clot material) can be accessed through a popliteal access site 560. The funnel catheter assembly 100 can extend from the popliteal access site 560 to a deployment position 562 in the blood vessel BV at which the funnel 120 (or the funnel 420) can be deployed. The deployment position 562 can be proximate to and proximal of the thrombus T. The funnel 120 can at least partially appose a wall of the blood vessel BV after the funnel catheter assembly 100 is moved from the first position to the second position to expand the funnel 120, as described in detail above with reference to FIGS. 1–4.

In the illustrated embodiment, a thrombus extraction device 564 has been (i) inserted through the funnel catheter assembly 100, (ii) passed through the thrombus T in the direction of blood flow, and (iii) expanded distal of the thrombus T. The thrombus extraction device 564 can include a coring element 565 (e.g., a stent-like device) and a capture element 566 (e.g., a braided mesh bag). In some embodiments, some or all of the thrombus extraction device 564 can extend into one of the iliac veins and/or the inferior vena cava. After expansion distal of the thrombus T, the thrombus extraction device 564 can be retracted through the thrombus T and into the lumen 108 of the inner shaft 104 (FIG. 1) through the funnel 120. During retraction, the coring element 565 can core/separate the thrombus T and the capture element 566 can capture all or a portion of the thrombus T. In some embodiments, the thrombus extraction device 564 and the associated thrombectomy procedure can be generally similar or identical to the thrombus extraction devices and associated methods described in detail in (i) U.S. Patent No. 9,700,332, filed September 16, 2016, and titled "INTRAVASCULAR TREATMENT OF VASCULAR OCCLUSION AND ASSOCIATED DEVICES, SYSTEMS, AND METHODS," and/or (ii) U.S. Patent No. 10,098,651, filed April 26, 2017, and titled "DEVICES AND METHODS FOR TREATING VASCULAR OCCLUSION," both of which are incorporated herein by reference in their entirety.

In one aspect of the present technology, as the thrombus extraction device 564 and the captured thrombus T are retracted through the funnel 120, the funnel 120 can capture/retain any of the thrombus T that breaks free of the thrombus extraction device 564 as the thrombus extraction device 564 is compressed into the inner shaft 104. Accordingly, the funnel 120 can inhibit portions of the thrombus T from traveling upstream where they could potentially embolize. In some embodiments, a vacuum (e.g., a pre-charged vacuum) can be applied to the inner shaft 104 (e.g., via a syringe coupled to the syringe connector 116 shown in FIG. 1) at any point during retraction of the thrombus extraction device 564. In some embodiments, application of the vacuum can generate instantaneous or nearly instantaneous suction at the distal portion 109b of the inner shaft 104 that can aspirate any remaining portions of the thrombus T into and/or through the inner shaft 104.

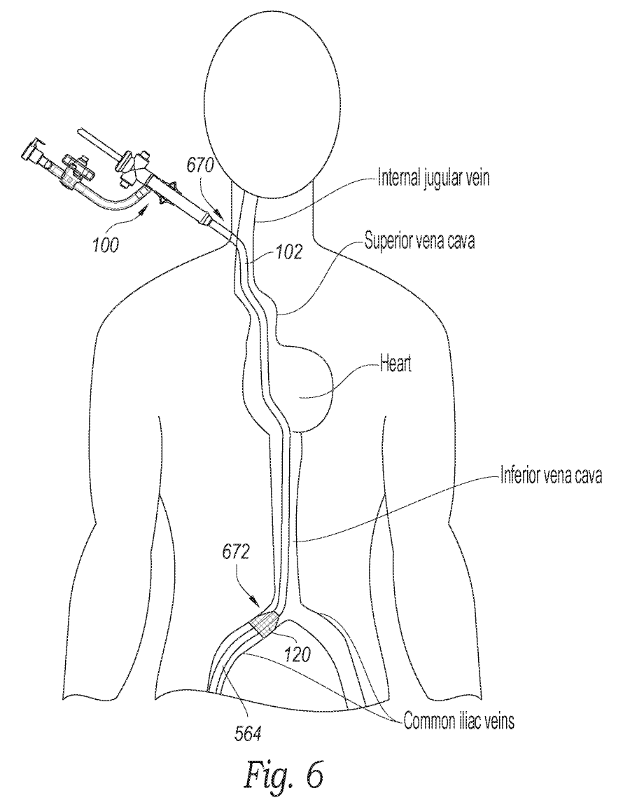

Referring next to FIG. 6, in some embodiments the thrombus T (not shown in FIG. 6; e.g., positioned lower in the iliac or femoral veins) can be accessed through an internal jugular access site 670, and the funnel catheter assembly 100 can be inserted into the patient's body via the internal jugular access site 670. The funnel catheter assembly 100 can extend from the internal jugular access site 670 to a deployment position 672 at which the funnel 120 (or the funnel 420) can be deployed proximal to the thrombus T. In the illustrated embodiment, the outer shaft 102 and the inner shaft 104 (obscured in FIG. 6) extend from the internal jugular access site 670 through the superior vena cava and the inferior vena cava to the deployment position 672 in one of the common iliac veins. In some embodiments, the deployment position 672 can be located in, for example, the inferior vena cava, one of the iliac veins, the femoral vein, the popliteal vein, before or beyond the iliac arch, or any other location proximate to and/or proximal to the thrombus T. In some embodiments, the thrombus extraction device 564 can be inserted through the funnel catheter assembly 100 and withdrawn through the thrombus T to capture the thrombus T, as described in detail above with reference to FIG. 5.

In one aspect of the present technology, accessing the thrombus T via the internal jugular access site 670 allows the funnel 120 to be positioned downstream of the thrombus T. Accordingly, the funnel 120 can capture any of the thrombus T that may break off and be carried downstream toward the heart during operation of the thrombus extraction device 564. In another aspect of the present technology, the funnel 120 can be compressed—for example, by moving the actuation member 134 from the second position to the first position as described in detail above with reference to FIGS. 1–4—before withdrawing the funnel catheter assembly 100 at the conclusion of the thrombectomy procedure. This prevents the funnel 120 from being withdrawn in the expanded configuration through the heart of the patient (e.g., through the right atrium) which could potentially damage the heart.

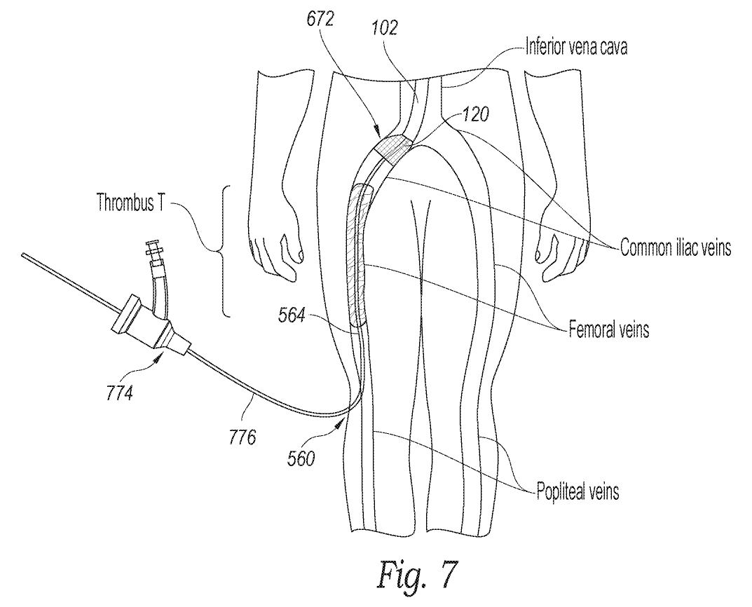

Referring next to FIG. 7, in some embodiments the thrombus T can be accessed by the thrombus extraction device 564 through the popliteal access site 560 using a separate introducer assembly 774, while the funnel catheter assembly 100 can be inserted into the patient's body via the internal jugular access site 670 (FIG. 6). The funnel 120 can be expanded at the deployment position 672 during a thrombectomy procedure using the thrombus extraction device 564. The funnel 120 can therefore capture any of the thrombus material T that may break away and flow downstream during the procedure. In the illustrated embodiment, the thrombus extraction device 564 is inserted through an outer shaft 776 of the introducer assembly 774. In one aspect of the present technology, in comparison to using the funnel catheter assembly 100 as the introducer for the thrombus extraction device 564, the outer shaft 776 can be made relatively larger than the inner shaft 104 because the outer shaft 102 need not be positioned thereabout for constraining the funnel 120. This can permit the thrombus extraction device 564 to be made larger and/or enable greater aspiration forces to be generated through the outer shaft 776.

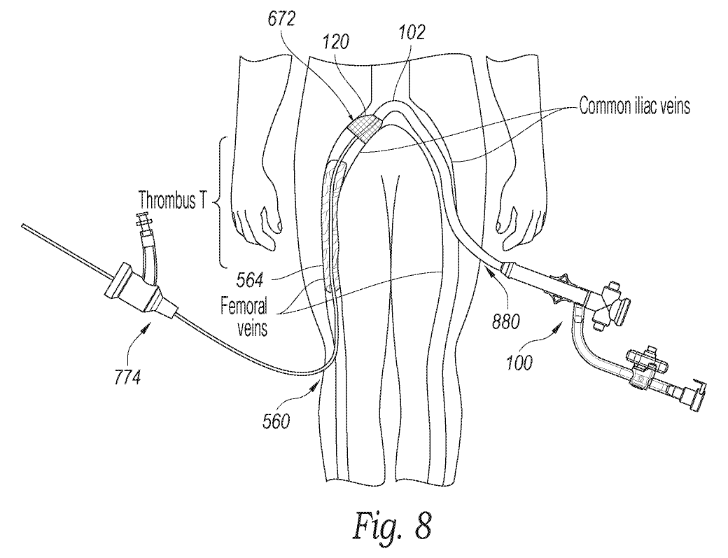

Referring next to FIG. 8, in some embodiments the thrombus T can be accessed by the thrombus extraction device 564 through the popliteal access site 560 using the introducer assembly 774, while the funnel catheter assembly 100 can be inserted into the patient's body via a femoral access site 880. The funnel catheter assembly 100 can traverse the common iliac veins to the deployment position 672, or another suitable deployment position.

In other embodiments, the funnel catheter assembly 100 can be inserted into a patient's body via other venous or arterial access sites, and can be used in a myriad of different procedures. For example, additional applications of the funnel catheter assembly 100 include but are not limited to:

Internal jugular vein (IJ) access for deployment in the inferior vena cava (IVC) for treatment of lower extremity deep vein thrombsis (DVT);

IJ access for deployment in the IVC for removal of IVC filters and/or treatment of thrombus located in the IVC;

IJ access for deployment in the deep veins of the lower extremity veins (iliac, femoral, popliteal veins, etc.) for treatment of lower extremity DVT;

Common femoral vein (CFV) access for deployment in the superior vena cava (SVC) for treatment of upper extremity DVT and/or SVC thrombus;

CFV access for deployment in the upper extremity veins (brachiocephalic, subclavian, axillary veins, etc.) for treatment of upper extremity DVT and/or SVC thrombus;

CFV access for deployment in the pulmonary arteries (PAs) for treatment of pulmonary embolism;

CFV access with transseptal access for deployment in the left atrium ostium and the left atrial appendage (LAA) for thrombus removal;

Internal Carotid Artery (ICA) accesss for deployment in the common carotid artery (CCA) for carotid endarterectomy (CEA);

ICA accesss for deployment in descending thoracic aorta for treatment of thoraco-abdominal and/or abdominal aortic (AA) thrombus and/or aorto-arterial thrombosis;

ICA accesss for deployment in the descending/thoracic aorta for treatment of renal thrombosis;

ICA accesss for intra-atrterial placement for treament of superior mesenteric artery (SMA) thrombosis (MAT);

Common Femoral Artery (CFA) access for treatment of atherosclerosis in the brachial artery, radial artery, popiteal artery, or dorsalis pedis artery;

CFA access for deployment in the common iliac artery for treatment of aorto-arterial thrombosis and/or occlusions;

CFA access for deployment in the abdominal aorta for treatment of renal artery thrombosis; and/or

CFA access for deployment in the aorta for treatment of thoraco-abdominal and/or AA thrombus and/or aorto-arterial thrombosis.

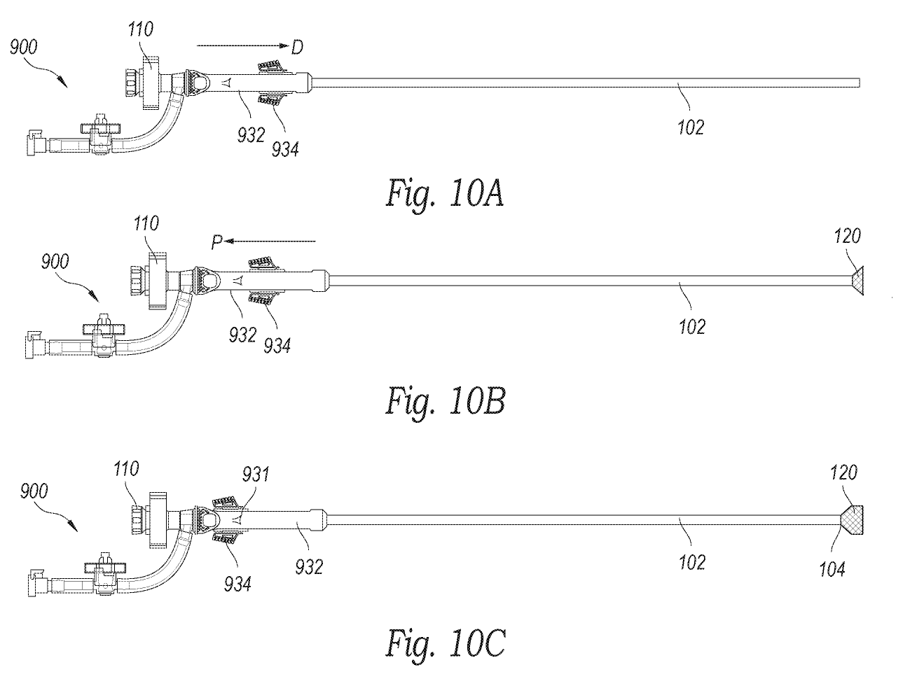

FIGS. 9A and 9B are enlarged, partially transparent side and side cross-sectional views, respectively, of a portion of a funnel catheter assembly 900 in accordance with additional embodiments of the present technology. Referring to FIGS. 9A and 9B together, the funnel catheter assembly 900 can include some features that are at least generally similar in structure and function, or identical in structure and function, to the corresponding features of the funnel catheter assembly 100 described in detail above with reference to FIGS. 1–8, and can operate in a generally similar or identical manner to the funnel catheter assembly 100. In the illustrated embodiment, for example, the funnel catheter assembly 900 includes the sealable hub 110, the connecting tube 114, the aspiration port 112, the outer shaft 102, and the inner shaft 104 (obscured in FIG. 9A). The funnel catheter assembly 900 further includes a control assembly 930 including a housing 932 and an actuation member 934 having a pair of grip member 938. As shown in FIG. 9B, the actuation member 934 includes a hub 936 positioned within the housing 932 and coupled to the outer shaft 102 (e.g., the proximal portion 107a of the outer shaft 102). The actuation member 934 is movable (e.g., slidable) relative to the housing 932 to advance/retract the outer shaft 102 relative to the inner shaft 104 and a funnel (e.g., the funnel 120 shown in FIGS. 10B and 10C) attached to a distal portion thereof.

The funnel catheter assembly 900 is in a first, sheathed position in FIG. 9A in which the funnel is constrained by the outer shaft 102 and in a second, unsheathed position in FIG. 9B in which the funnel is not constrained by the outer shaft 102. Referring to FIG. 9A, in some embodiments the housing 932 can include a marking 931 indicating a position of the actuation member 934 in which the funnel is in the unsheathed position. In some embodiments, a proximal portion 933a of the housing 932 can include gripping features, such as a groove 935, a cross-hatched pattern 937, and/or other features for increasing the grip-ability of the housing 932. In some embodiments, an operator can grip the housing 932 (e.g., via the groove 935 and/or the pattern 937) while moving (i) the actuation member 934 relative to the housing 932 and/or (ii) the housing 932 relative to the actuation member 934. In some embodiments, the funnel catheter assembly 900 can further include a lock mechanism 911 that can be attached to the sealable hub 110 (e.g., to buttons thereof) to lock the sealable hub 110 in the open/unsealed position.

In the illustrated embodiment, the funnel catheter assembly 900 further includes a rotatable side port 950 fluidly connecting the sealable hub 110, the connecting tube 114, and the inner shaft 104. FIG. 9C is an enlarged isometric cross-sectional view of a portion of the sealable hub 110, the rotatable side port 950, and the control assembly 930 in accordance with embodiments of the present technology. Referring to FIG. 9C, the side port 950 defines a lumen 952 that provides a fluid path from the inner shaft 104 to the sealable hub 110. In the illustrated embodiment, the side port 950 includes a first groove 954 and a second groove 956 that each extend circumferentially about an outer surface of the side port 950. The sealable hub 110 includes a first projection 964 positioned in the first groove 954 and the housing 932 includes a second projection 966 positioned in the second groove 956. The engagement of the first and second projections 964, 966 in the first and second grooves 954, 956 couples the housing 932 to the sealable hub 110. Moreover, the first and second projections 964, 966 are rotatable within the first and second grooves 954, 956 such that the sealable hub 110, the side port 114, and the housing 932 can each rotate independently of one another while remaining coupled together.

In some embodiments, the control assembly 930 can further include a connector 958 coupled to the proximal portion 109a of the inner shaft 104 and positioned at least partially between the side port 950 and the housing 932. The connector 958 can be rotatable within the housing 932 such that the housing 932 can rotate independently of the connector 958 and the inner shaft 104. Accordingly, in some aspects of the present technology rotation of the sealable hub 110, the side port 950 (e.g., the aspiration port 112 shown in FIGS. 9B and 9C), and/or the housing 932 will not substantially rotate the inner shaft 104. This can help inhibit the funnel attached to the inner shaft 104 from rotating within a vessel during a procedure using the funnel catheter assembly 900—while still allowing for movement of the sealable hub 110, the side port 950, and/or other components of the funnel catheter assembly 900.

FIGS. 10A–10C are side views of the funnel catheter assembly 900 in the sheathed position, a partially-unsheathed or intermediate position, and the unsheathed position, respectively, in accordance with embodiments of the present technology. Referring to FIGS. 10A–10C together, the funnel catheter assembly 900 can be moved between the sheathed and unsheathed positions by moving the actuation member 934 relative to the housing 932 and/or by moving the housing 932 (and the coupled sealable hub 110) relative to the actuation member 934. In some embodiments, for example, the operator can slide the actuation member 134 relative to the housing 932 in the direction indicated by the arrow P in FIG. 10B to retract the outer shaft 102 to unsheathe the funnel 120. In some aspects of the present technology, moving the actuation member 934 rather than the housing 932 moves only the outer shaft 102 such that the funnel 120 remains in a constant or generally constant position (e.g., stationary position). In other embodiments, the operator can advance the housing 932 relative to the actuation member 934 in the direction indicated by the arrow D in FIG. 10A to unsheathe the funnel 120. In some aspects of the present technology, moving the housing 932 relative to the actuation member 934 advances the inner shaft 104 and the funnel 120 distally relative to the outer shaft 102.

In the unsheathed position shown in FIG. 10C, the actuation member 934 can be positioned adjacent or near the marking 931 to indicate to the operator that the funnel 120 is deployed. In some embodiments, to re-sheathe the funnel 120 (e.g., from the unsheathed position shown in FIG. 10C to the sheathed position shown in FIG. 10A), the operator can retract the housing 932 relative to the actuation member 934 (e.g., in the direction of the arrow P) to pull the funnel 120 into the outer shaft 102. In some aspects of the present technology, such a retracting motion can be intuitive to the user as the motion of the housing 932 is in the same direction as the removal/collapse of the funnel 120. Nevertheless, in other embodiments the operator can advance the actuation member 934 relative to the housing 932 to sheathe the funnel 120 within the outer shaft 102.

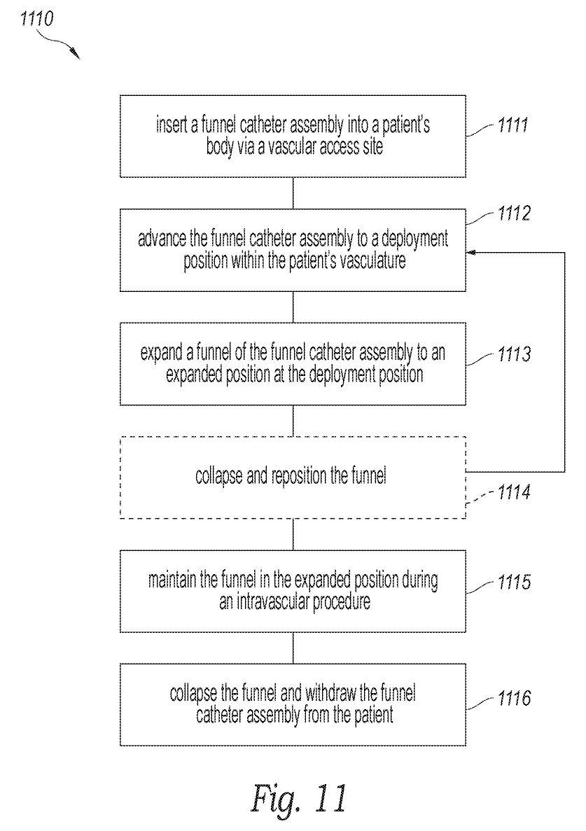

FIG. 11 is a flow diagram of a process or method 1110 for operating the funnel catheter assembly 100 and/or the funnel catheter assembly 900 (collectively referred to as "the funnel catheter assembly") during an intravascular procedure in accordance with embodiments of the present technology. Although some features of the method 1110 are described in the context of the embodiments shown in FIGS. 1–10C for the sake of illustration, one skilled in the art will readily understand that the method 1110 can be carried out using other suitable systems and/or devices described herein.

At block 1111, the method 1110 includes inserting the funnel catheter assembly into a patient's body via a vascular site. For example, the dilator 350, the outer shaft 102, and inner shaft 104 can be inserted together through a venous or arterial access site.

At block 1112, the method 1110 includes advancing the funnel catheter assembly to a deployment position within the vasculature of the patient. For example, the dilator 350, the outer shaft 102, and inner shaft 104 can be advanced together through the vasculature to the selected deployment position. The funnel catheter assembly can be advanced in the first position such that the funnel 120 is constrained/compressed within the outer shaft 102. The deployment position can be a portion of a blood vessel, a portion of the heart, or another suitable location.

At block 1113, the method 1110 includes expanding the funnel 120 at the deployment position. For example, the control assembly 130 of the funnel catheter assembly 100 can be moved from the first position to the second position to release/unsheathe the funnel 120 from within the outer shaft 102, thereby allowing the funnel 120 to expand at the deployment position. Likewise, the actuation member 934 can be slid relative to the housing 932 to unsheathe the funnel 120. After expansion, the funnel 120 can appose/contact the anatomy surrounding the deployment position, such as a wall of a blood vessel. In some embodiments, the dilator 350 can be removed from the funnel catheter assembly before expanding the funnel 120.

At block 1114, the method 1110 optionally includes compressing and repositioning the funnel 120. For example, the control assembly 130 can be moved from the second position to the first position to constrain/sheathe the funnel 120 within the outer shaft 102. Likewise, the housing 932 of the funnel catheter assembly 900 can be retracted proximally relative to the actuation member (and/or the actuation member 934 can be advanced distally relative to the housing) to sheathe the funnel 120. Then, the funnel catheter assembly can be repositioned to a different deployment position (block 1112) and expanded once again (block 1113). In some embodiments, the dilator 350 can be reinserted into the funnel catheter assembly 100 before repositioning the funnel catheter assembly 100.

At block 1115, the method 1110 includes maintaining the funnel 120 in the expanded position during an intravascular procedure. As described in detail above, the funnel 120 can capture thrombi that break free during the intravascular procedure to inhibit their embolization elsewhere in the vasculature of the patient.

At block 1116, the method 1110 includes compressing the funnel 120 and withdrawing the funnel catheter assembly 100 from the patient. For example, the control assembly 130 of the funnel catheter assembly 100 can be moved from the second position to the first position to constrain/sheathe the funnel 120 within the outer shaft 102, and the funnel catheter assembly 100 can then be withdrawn proximally to and from the vascular access site. Likewise, the housing 932 of the funnel catheter assembly 900 can be retracted proximally relative to the actuation member (and/or the actuation member 934 can be advanced distally relative to the housing) to sheathe the funnel 120.

### Examples

Several aspects of the present technology are set forth in the following examples:

1. A funnel catheter assembly, comprising:

an outer shaft defining a lumen;

an inner shaft extending through the lumen and having a proximal portion and a distal portion;

an expandable funnel coupled to the distal portion of the inner shaft; and

a control assembly configured to move the funnel between a first position and a second position, wherein—

in the first position, the funnel is constrained within the lumen of the outer shaft, and

in the second position, the funnel is positioned at least partially outside the lumen of the outer shaft such that the funnel can expand.

2. The funnel catheter assembly of example 1 wherein the control assembly is coupled to the outer shaft and configured to move the outer shaft relative to the inner shaft.

3. The funnel catheter assembly of example 1 or example 2 wherein the control assembly includes an actuator movable to move the funnel between the first and second positions.

4. The funnel catheter assembly of example 3 wherein the actuator is a slider.

5. The funnel catheter assembly of any one of examples 2–4 wherein the funnel includes a proximal portion and a distal portion, and wherein the proximal portion of the funnel is coupled to the distal portion of the inner shaft.

6. The funnel catheter of example b wherein the distal portion of the funnel is coupled to the outer shaft.

7. The funnel catheter assembly of any one of examples 1–6, further comprising a sealable hub and a side port, wherein the side port is rotatably coupled between the control assembly and the sealable hub.

8. A funnel catheter assembly, comprising:

an outer shaft defining an outer lumen;

an inner shaft extending through the outer lumen and having a proximal portion and a distal portion;

an expandable funnel coupled to the distal portion of the inner shaft; and

a control assembly operably coupled to the proximal portion of the outer shaft and configured to move the outer shaft between a first position and a second position, wherein—

in the first position, the outer shaft is positioned at least partially over the funnel to radially constrain the funnel, and

in the second position, the outer shaft is retracted proximally relative to the funnel such that the funnel can radially expand.

9. The funnel catheter assembly of example 8 wherein the control assembly includes a housing and an actuation member, wherein the actuation member is coupled to a proximal portion of the outer shaft, and wherein the actuation member is slidable along the housing to move the outer shaft between the first and second positions.

10. The funnel catheter assembly of example b or example 9 wherein the funnel is configured to self-expand.

11. The funnel catheter assembly of example b or example 9 wherein the funnel is non-self-expanding, wherein the funnel is further coupled to a distal portion of the outer shaft, and wherein movement of the outer shaft from the first position to the second position is configured to expand the funnel.

12. The funnel catheter assembly of example 11 wherein a distal portion of the funnel is coupled to the distal portion of the outer shaft via one or more tethers.

13. The funnel catheter assembly of any one of examples 8–12 wherein the inner shaft defines an inner lumen sized to receive a dilator.

14. A method of operating a funnel catheter assembly during an intravascular procedure on a patient, the method comprising:

at least partially inserting an inner shaft, an outer shaft, and a funnel of the funnel catheter assembly into the vasculature of the patient;

advancing the inner shaft, the outer shaft, and the funnel together to a deployment position within the vasculature of the patient, wherein the funnel is sheathed within a lumen of the outer shaft during the advancement;

moving the outer shaft relative to the inner shaft to unsheathe the funnel and permit the funnel to expand to an expanded position;

maintaining the funnel in the expanded position during at least a portion of the intravascular procedure;

moving the outer shaft relative to the inner shaft and/or moving the inner shaft relative to the outer shaft to sheath the funnel within the lumen of the outer shaft; and

withdrawing the funnel catheter assembly from the patient.

15. The method of example 14 wherein the funnel is self-expandable, and wherein moving the outer shaft relative to the inner shaft to unsheathe the funnel includes permitting the funnel to self-expand to the expanded position.

16. The method of example 14 or example 15 wherein moving the outer shaft relative to the inner shaft to unsheathe the funnel includes moving a slider of a control assembly of the funnel catheter assembly from a first position to a second position, and wherein the slider is coupled to a proximal portion of the outer shaft.

17. The method of any one of examples 14–16 wherein moving the outer shaft relative to the inner shaft to unsheathe the funnel includes rotating a rotatable element of a control assembly of the funnel catheter assembly from a first position to a second position.

18. The method of any one of examples 14, 16, and 17 wherein the funnel is non-self-expanding, and wherein the method further comprises actuating the funnel to expand to the expanded position.

19. The method of any one of examples 14–18 wherein—

inserting the inner shaft, the outer shaft, and the funnel into the vasculature of the patient further includes at least partially inserting a dilator positioned within the inner shaft into the vasculature of the patient; and

advancing the inner shaft, the outer shaft, and the funnel further includes advancing the inner shaft, the outer shaft, the funnel, and the dilator together to the deployment position.

20. The method of any one of examples 14–19 wherein moving the outer shaft relative to the inner shaft to unsheathe the funnel includes moving the outer shaft in a first direction, and wherein the method further comprises moving the outer shaft relative to the inner shaft to sheath the funnel by moving the outer shaft in a second direction opposite to the first direction.

### Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word "or" is expressly limited to mean only a single item exclusive from the other items in reference to a list of two or more items, then the use of "or" in such a list is to be interpreted as including (a) any single item in the list, (b) all of the items in the list, or (c) any combination of the items in the list. Additionally, the term "comprising" is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A funnel catheter assembly, comprising:
an outer shaft defining a lumen;
an inner shaft extending through the lumen and having a proximal portion and a distal portion;
an expandable funnel coupled to the distal portion of the inner shaft; and
a control assembly configured to move the funnel between a first position and a second position, wherein—
in the first position, the funnel is constrained within the lumen of the outer shaft, and
in the second position, the funnel is positioned at least partially outside the lumen of the outer shaft such that the funnel can expand.

2. The funnel catheter assembly of claim 1 wherein the control assembly is coupled to the outer shaft and configured to move the outer shaft relative to the inner shaft.

3. The funnel catheter assembly of claim 1 wherein the control assembly includes an actuator movable to move the funnel between the first and second positions.

4. The funnel catheter assembly of claim 3 wherein the actuator is a slider.

5. The funnel catheter assembly of claim 1 wherein the funnel includes a proximal portion and a distal portion, and wherein the proximal portion of the funnel is coupled to the distal portion of the inner shaft.

6. The funnel catheter of claim 5 wherein the distal portion of the funnel is coupled to the outer shaft.

7. The funnel catheter assembly of claim 1, further comprising a sealable hub and a side port, wherein the side port is rotatably coupled between the control assembly and the sealable hub.

8. A funnel catheter assembly, comprising:
an outer shaft defining an outer lumen;
an inner shaft extending through the outer lumen and having a proximal portion and a distal portion;
an expandable funnel coupled to the distal portion of the inner shaft; and
a control assembly operably coupled to the proximal portion of the outer shaft and configured to move the outer shaft between a first position and a second position, wherein—
in the first position, the outer shaft is positioned at least partially over the funnel to radially constrain the funnel, and
in the second position, the outer shaft is retracted proximally relative to the funnel such that the funnel can radially expand.

9. The funnel catheter assembly of claim 8 wherein the control assembly includes a housing and an actuation member, wherein the actuation member is coupled to a proximal portion of the outer shaft, and wherein the actuation member is slidable along the housing to move the outer shaft between the first and second positions.

10. The funnel catheter assembly of claim 8 wherein the funnel is configured to self-expand.

11. The funnel catheter assembly of claim 8 wherein the funnel is non-self-expanding, wherein the funnel is further coupled to a distal portion of the outer shaft, and wherein movement of the outer shaft from the first position to the second position is configured to expand the funnel.

12. The funnel catheter assembly of claim 11 wherein a distal portion of the funnel is coupled to the distal portion of the outer shaft via one or more tethers.

13. The funnel catheter assembly of claim 8 wherein the inner shaft defines an inner lumen sized to receive a dilator.

14. A method of operating a funnel catheter assembly during an intravascular procedure on a patient, the method comprising:
at least partially inserting an inner shaft, an outer shaft, and a funnel of the funnel catheter assembly into the vasculature of the patient;
advancing the inner shaft, the outer shaft, and the funnel together to a deployment position within the vasculature of the patient, wherein the funnel is sheathed within a lumen of the outer shaft during the advancement;
moving the outer shaft relative to the inner shaft to unsheathe the funnel and permit the funnel to expand to an expanded position;
maintaining the funnel in the expanded position during at least a portion of the intravascular procedure;
moving the outer shaft relative to the inner shaft and/or moving the inner shaft relative to the outer shaft to sheath the funnel within the lumen of the outer shaft; and
withdrawing the funnel catheter assembly from the patient.

15. The method of claim 14 wherein the funnel is self-expandable, and wherein moving the outer shaft relative to the inner shaft to unsheathe the funnel includes permitting the funnel to self-expand to the expanded position.

16. The method of claim 14 wherein moving the outer shaft relative to the inner shaft to unsheathe the funnel includes moving a slider of a control assembly of the funnel catheter assembly from a first position to a second position, and wherein the slider is coupled to a proximal portion of the outer shaft.

17. The method of claim 14 wherein moving the outer shaft relative to the inner shaft to unsheathe the funnel includes rotating a rotatable element of a control assembly of the funnel catheter assembly from a first position to a second position.

18. The method of claim 14 wherein the funnel is non-self-expanding, and wherein the method further comprises actuating the funnel to expand to the expanded position.

19. The method of claim 14 wherein—
inserting the inner shaft, the outer shaft, and the funnel into the vasculature of the patient further includes at least partially inserting a dilator positioned within the inner shaft into the vasculature of the patient; and
advancing the inner shaft, the outer shaft, and the funnel further includes advancing the inner shaft, the outer shaft, the funnel, and the dilator together to the deployment position.

20. The method of claim 14 wherein moving the outer shaft relative to the inner shaft to unsheathe the funnel includes moving the outer shaft in a first direction, and wherein the method further comprises moving the outer shaft relative to the inner shaft to sheath the funnel by moving the outer shaft in a second direction opposite to the first direction.
